(19) United States
(12) Patent Application Publication
(10) Pub. No.: US 2005/0009813 A1
(43) Pub. Date: Jan. 13, 2005

(54) Use of desoxypeganine for treating clinical depression

(76) Inventors: Joachim Moormann, Werne (DE); Hermann Mucke, Wien (AT)

Correspondence Address: BIRCH STEWART KOLASCH & BIRCH, PO BOX 747, FALLS CHURCH, VA, 22040-0747, US

(21) Appl. No.: 10/496,366

(22) PCT Filed: Dec. 14, 2002
(86) PCT No.: PCT/EP02/14274

(30) Foreign Application Priority Data
Dec. 21, 2001 (DE) 101 63 667.9

(51) Int. Cl. A61K 31/55

(52) U.S. Cl. 514/221

(57) ABSTRACT

Use of deoxypeganine, as a free base or as acid addition salt, for the production of drugs intended for the therapy of clinical depression, especially of depression connected with dementia or abuse of alcohol and/or nicotine.

The present invention relates to the use of deoxypeganine for the production of drugs intended for the therapy of clinical depression, especially of depression in connection with dementia or alcohol and/or nicotine abuse.

Unipolar depression (by contrast to the bipolar disorder, formerly called manic-depressive disorder) according to the International Classification of Diseases (ICD-10) and the American Diagnostic and Statistics Manual (DSM-IV) is a syndrome characterized by a combination of depressed frame of mind, general lack of drive and interest with, frequently simultaneous, restlessness, sleeping disorders and social withdrawal. Apart from the suffering of the persons concerned and of the risk of suicide, depression—on account of therapy costs and inability to work—causes a hardly estimable overall damage to the national economy, which, for instance, in 1990 probably amounted to about 43.7 thousand million dollars in the USA.

Depression is by far the most frequent psychic disease. A synopsis of hundreds of large epidemiological studies shows that 10-25% of all women and 5-12% of all men suffer from depression at least once in their life. In the industrialised states about 5% of the population suffer from depression at any time; here, one has to assume that 15-25% of all patients who go to a general physician or to a hospital suffer from depression. Worldwide, this applies to about every tenth of such patients. Depression is a disease with a high and progressive relapse proportion, which is moreover continuously on the increase. The probability of a relapse increases from 50% after a depressive episode to 70% after two, and 90% after three such episodes. WHO assumes that in 2020, 5.7% of the disease burden on the world population will be on account of depression—a proportion that is only little behind that of cardiovascular diseases. Based on this information one can calculate that at any time 150 million people are suffering from clinical depression (Mucke HAM.: Next-Generation CNS Therapeutics. Decision Resources, Inc. [Waltham, Mass., USA], 2001].

There is a three-way comorbidity, proven in numerous studies, between depression, alcohol abuse and nicotine abuse (see, for example, J. Hamalainen et al.; J. Epidemiol. Community Health 2001; 55(8): 573-576). Traumatic experiences and chronic stress—two essential etiological factors of depression—are also significantly connected with the forming of consumption behaviour with respect to alcohol and nicotine (H. J. Little, Alcohol Res. Health 2000; 24(4): 215-224).

The most effective drugs for the therapy of clinical depression are the so-called “tricyclics”—tricyclic compounds, which both block neuronal receptors for serotonine and norepinephrine and inhibit the reabsorption of these neurotransmitters in the respective neurons, and thereby tend to normalise their intrasynaptic concentration, which concentration is reduced in case of depression. Tricyclics are still being widely used today although their use is connected with considerable side effects, particularly of the cardiovascular type.

Selective serotonine-reabsorption inhibitors (SSRIs) came to be used from around the mid-1980s. Seen as a class, SSRIs are not as highly effective as typical tricyclics and begin to show full action only after a delay of 1-2 weeks, but their side effects are considerably smaller and on account of their much lower acute toxicity it is virtually impossible to commit suicide with SSRIs. For this reason, SSRIs have frequently become the antidepressants of first choice.

However, apart from the side effects, both tricyclics and SSRIs leave considerable gaps in the therapy of depression: About 30% of all patients do not respond to either of the two classes of antidepressants in an adequate fashion (so-called therapy-refractory depression). Furthermore, there is no single active substance that would be able to treat the problem of a drinking depressive patient both at the level of depression as well as by means of reduction of alcohol abuse. For the heavy-smoking depressive patient, there has so far been only a single active agent (Bupropion, GlaxoSmithKline) available, however, only in the form of two separate and differently dosed medicaments of which one (Wellbutrin®) is approved only for the treatment of depression, and the other (Zyban®) exclusively for supporting the withdrawal treatment of smokers.

An alternative to tricyclics and SSRIs are mono-amino oxidase inhibitors (MAOIs). These are a class of active agents which has been known for about 50 years and which by inhibiting the degradation of all “monoamine neurotransmitters” (i.e. including dopamine) increase the concentration thereof in the brain. The early active agents of this class inhibit both subtypes of monoamine oxidase (A and B), partly in an irreversible manner. Owing to the occurrence of liver damage on the one hand and of the “cheese effect” (a hypertensive crisis triggered by a blockage of the degradation of tyramine taken in with food stuffs such as, for example, cheese) on the other hand, MAOI's were dropped in favour of tricyclics.

Consequently, there still exists a considerable need for antidepressants, especially those which are better suited than commercial active agents both for the therapy of refractory depression as well as in the specific situation of depressive patients abusing alcohol and/or nicotine.

The object of the present invention therefore was to provide a drug for the therapy of dementia, especially of refractory dementia, which drug is, however, better suited—even in the specific situation of depressive patients abusing alcohol and/or nicotine—than commercial active agents, but which does not have the aforementioned disadvantages.

Deoxypeganine (1,2,3,9-tetrahydropyrrolo[2,1-b]quinazoline) is an alkaloid of molecular formula C₁₁H₁₂N₂ which is present in plants of the Zygophyllaceae family. Deoxypeganine is preferably obtained by isolation from Syrian rue (Peganum harmala) or by chemical synthesis. It is known to the pharmaceutical art from the literature and, in particular, from patent specifications.

DE-A 199 06 978, respectively WO 00/48582, describes drugs based on deoxypeganine for the therapy of drug addiction and drug dependence.

DE-A 199 06 975, respectively WO 00/48599, describes the use of deoxypeganine for the therapy of Alzheimer's dementia.

DE-A 199 06 979, respectively WO 00/48445, describes drugs based on deoxypeganine for the therapy of nicotine dependence.

On the basis of its pharmacological properties, deoxypeganine is included in the group of reversibly acting cholinesterase inhibitors. The fact that deoxypeganine does not only inhibit acetylcholinesterase but also mono-amino oxidases, is in general terms known from these publications, but these documents do not distinguish between the two subtypes mono-amino oxidase A and B in any way. Above all, mono-amino oxidase inhibition is constantly described as a merely complementary action which is intended to reinforce the acetylcholinesterase inhibition of deoxypeganine, the latter inhibition being regarded as the most important; it is, for instance, expressly mentioned that the advantage of the simultaneous inhibition of acetylcholinesterase and mono-amino oxidase compensates for the—relative to the unit of weight—lower cholinesterase inhibition (compared to the prototypically potent cholinesterase inhibitor physostigmine) with respect to the respective applications claimed. Finally, none of these documents addresses depression as a possible field of application.

In the course of further pharmacological studies, it has now been found that, surprisingly, deoxypeganine does indeed inhibit acetylcholinesterase, as described in the above-mentioned documents, but the quantitative main action in vitro consists in a selective inhibition of mono-amino oxidase of type A (MAO-A), whereas the Type B enzyme is not significantly inhibited. The mentioned side-effects of the early mono-amino oxidase inhibitors can be largely avoided by selective, reversible inhibitors of mono-amino oxidase A (RIMA).

Furthermore, it was found that deoxypeganine in an appropriate animal experiment shows strongly antidepressive and psychomotorically stimulating activities—this finding being related to the above double finding, but being totally surprising with respect to the state of the art. The maximal action here occurs already at dosages that in an animal model of cholinergic activation do not yet show statistically significant effects.

The inhibitory action of deoxypeganine in respect of monoamino oxidase A from rat brain (Wistar strain) was measured in the concentration range of from 10 nM to 10 μM according to the method described by Medvedev et al. (Biochem Pharmacol 1994; 47(2): 303-308) and compared with clorogyline as the positive control, whereby in both cases 95 μM [³H]serotonine in a solution of 1% dimethylsulfoxide in 20 mM of potassium dihydrogenphosphate buffer pH 7.4 served as substrate. For deoxypeganine a value of 1.49 μM resulted for the semi-maximal inhibitory action (inhibitory concentration 50%=IC₅₀). This value is almost a power of ten below the IC₅₀ value obtained for the inhibition of acetylcholinesterase in another in vitro system. Monoamino oxidase B, by contrast, was subject to an inhibition of only 15-20% at a concentration of 10 μM.

To verify whether this MAO-A-inhibitory action in vivo is relevant, deoxypeganine was tested in the “Forced Swimming Test” on rats (R. D. Porsolt et al., Nature 1977; 266(5604): 730-732). This model is based on the behaviour called “behavioural despair” which shows the animals in a hopeless situation already known to them: If they are placed in a vessel filled with water, from which they cannot escape, they will after a certain period abandon their attempts at escape and will only make the most indispensable swimming movements. The time spent with attempts at escape until the animals fall into this psychomotorical inactivation (considered to be a surrogate for depression) is measured; prolonged activity corresponds to antidepressive action.

In the concrete case, 50 male rats, about 6 weeks old (strain Sprague Dawley); acquired from Charles River UK Ltd.) were divided into 7 groups with 10 animals each. On the first day, after termination of the habituation phase, each animal was placed for 10 minutes in a cylindrical vessel filled about 15 cm high with 25°-warm water; this was followed, after one hour, 19 hours, and 23 hours, by three oral administrations of (depending on the group) either negative control (water), 15 mg/kg positive control (imipramine.HCl), or deoxypeganine.HCl in dosages of 1.0, 2.5, 7.5, 15.0 or 22.5 mg/kg of bodyweight. The volume, administered with the aid of a gastric tube, was in each case 5 ml/kg of bodyweight. One hour after the third of these treatments, each animal was again placed in the vessel for exactly 5 minutes and the time spent in minimal mobility was measured.

The potent tricyclic antidepressant imipramine, which according to the results of preliminary tests defines the maximally achievable action in this system for the mentioned dose of 15 mg/kg, lowered the time spent in minimal mobility as compared to water by 56.5% up to 58.9%. While deoxypeganine at a dose of 1 mg/kg was not yet effective, and at 2.5 mg/kg was only partially effective, with all concentrations from 7.5 mg/kg reductions in the range of, on average, 41.4% to 44.1% were achieved. All of these plateau values were statistically significant at the level p<0.01. Thus, for all of the concentrations the effect of the treatment with deoxypeganine indeed remained below the maximum that is possible in this system, but already at half the dose (7.5 mg/kg) used for the positive control, the maximum value for this substance was reached (see Table 1).

Action of deoxypeganine HCl in the Porsolt Swimming Test (rat, SD strain)

| | Dose | Acquired Immobility | Change |
|---|---|---|---|
| | (mg/kg | (group mean value in | relative to |
| Treatment | p.o.) | minutes ± standard dev.) | control group |
| negative control | — | 3.45 ± 0.60 | — |
| (water f. injection) | | 3.38 ± 0.60 | |
| deoxypeganine | 1.0 | 3.46 ± 0.83 | +0.3% |
| hydrochloride | | | |
| deoxypeganine | 2.5 | 2.80 ± 1.02 | −18.8% |
| hydrochloride | | | |
| deoxypeganine | 7.5 | 1.82** ± 0.96 | −47.2% |
| hydrochloride | | 1.98** ± 0.83 | −41.4% |
| deoxypeganine | 15.0 | 1.90** ± 0.59 | −44.1% |
| hydrochloride | | | |
| deoxypeganine | 22.5 | 1.90** ± 0.54 | −43.8% |
| hydrochloride | | | |
| positive control | 15 | 1.50** ± 0.49 | −56.5% |
| (imipramine HCl | | 1.39** ± 0.52 | −58.9% |
| in max. | | | |
| effective dose) | | | |

**p < 0.01

Since such results can in principle also be obtained by psychomotorically activating substances which have no anti-depressive action, the effect of deoxypeganine was also examined, with the same dose range, in the so-called Open Field Paradigma. Here one makes use of the fact that it is stressful for rats to stay in an open, bright area, and therefore such situations are avoided by the rats if possible. Such an experiment, likewise performed using Sprague-Dawley rats, involving treatment continuing for 2 weeks with daily deoxypeganine doses of from 2.5 to 22.5 mg/kg, yielded no indication of psychomotoric activation, however, so that the result of the Porsolt test (maximal action, comparable to the antidepressant imipramine, at 7.5 mg/kg p.o.) must be interpreted as meaningful.

This is all the more astonishing as a further test system—wherein by making use of the acetylcholinesterase-inhibiting action of deoxypeganine, the cholinergic memory deficits in rats with partially destroyed central cholinergic pathways were compensated—showed a clear linear dependence in the same dose range which at an oral dose of 7.5 mg/kg was not yet statistically significant and at 22.5 mg/kg had not yet reached its maximum value. It can be inferred therefrom that the effect observable in the swimming test reaches its maximum at a fraction of the dose necessary for maximal activation of the cholinergic system, which according to the state of the art could not be reckoned with.

Thus, in a recognized animal model of depression, deoxypeganine has antidepressive, respectively psychomotorically activating action, namely at a maximal degree already at a dose which in a behaviour model of cholinergic compensation still shows absolutely suboptimal action under otherwise equal conditions.

Deoxypeganine is therefore potentially suitable as an anti-depressant.

The administration of deoxypeganine may be peroral or parenteral. For oral administration, known administration forms can be used, such as tablets, capsules, coated tablets, lozenges.

Also suitable are liquid or semiliquid dosage forms, for example as drinking solutions, in which case the agent is present in the form of a solution or suspension. Solvents or suspending agents which can be used are water, aqueous media or pharmacologically acceptable oils (vegetable or mineral oils).

The deoxypeganine-containing drugs are preferably formulated as depot drugs which are able to deliver this agent to the body in a controlled manner over a prolonged period.

Moreover, deoxypeganine may according to the invention also be administered rectally (e.g. by introducing suppositories), inhalationally (by breathing in aerosols with defined concentration and size distribution of the particles), transdermally (by active agent-containing patches, liniment solutions, gels etc.), transmucosally (in the sense of an absorption through the oral and nasal mucous membranes, with the active agent being released in the oral cavity by dissolution in saliva or being brought into the nose by spray solutions and the like), by means of implanted vessels (which release the active agent passive-osmotically or controlled by means of minipumps or the like), by intravenous, intramuscular or subcutaneous injection and intracerebroventricularly.

In connection with the parenteral administration, it is with particular advantage possible to use transdermal or transmucosal dosage forms for the deoxypeganine administration according to the invention, in particular adhesive transdermal therapeutic systems (agent plasters) as described specifically for deoxypeganine in DE-A 199 06 977. These make it possible to deliver the agent in a controlled manner over a prolonged period via the skin to the patient to be treated.

According to the invention, deoxypeganine can be used both in the form of its free base and as acid addition salt for treatment; preferred salts are deoxypeganine hydrochloride and deoxypeganine hydrobromide. [In addition, it is also possible to use salts of other pharmacologically acceptable acids, e.g. citrate, tartrate or acetate.

The pharmaceutical preparations which are used according to the present invention for administering deoxypeganine may contain one or more of the following additives:
antioxidants, synergists, stabilizers;
preservatives;
taste corrigents;
colours;
solvents, solubilizers;
surfactants (emulsifiers, solubilizers, wetting agents, antifoams);
agents influencing viscosity and consistency, gel formers;
absorption promoters;
adsorbents, humectants, glidants;
agents influencing disintegration and dissolution, fillers (extenders), peptisers;
release-delaying agents.

This list is not definitive; the suitable physiologically acceptable substances are known to the skilled person.

Deoxypeganine is preferably administered in a pharmaceutical preparation which contains the agent in proportions of from 0.1 to 90% by weight, particularly preferably in proportions of from 2 to 20% by weight, in each case calculated as free deoxypeganine. The deoxypeganine-containing pharmaceutical preparations used according to the invention may additionally contain the additives, such as inactive ingredients, excipients, vehicles and/or stabilizers, in the amounts known to the skilled person.

The dose administered each day is preferably in the range from 0.1 to 100 mg, in particular from 10 to 50 mg. It should be adjusted appropriately depending on the individual requirements.

1. A method for treating a subject presenting clinical depression comprising administering to said subject an amount of deoxypeganine, as a free base or as acid addition salt, effective for treating clinical depression.

2. The method according to claim 1, characterized in that the said depression is a therapy-refractory depression.

3. The method according to claim 1 or 2, characterized in that the said depression is connected with dementia.

4. The method according to claim 1 or 2, characterized in that the depression is connected with the abuse of addictive substances or narcotics.

5. The method according to claim 1 or 2, characterized in that the said addictive substance abuse is alcohol and/or nicotine abuse.

6. The method according to claim 1 or 2, characterized in that the administered dose is in the range of 0.1 to 100 mg, preferably 10 to 50 mg, per day.

7. The method according to claim 1 or 2, characterized in that deoxypeganine is administered in a pharmaceutical preparation which contains the active agent in proportions from 0.1 to 90%-wt., preferably 2 to 20%-wt., calculated as free deoxypeganine.

8. The method according to claim 7, characterized in that deoxypeganine is administered in a pharmaceutical preparation with depot action.

9. The method according to claim 7, characterized in that deoxypeganine is administered orally.

10. The method according to claim 7, characterized in that deoxypeganine is administered parenterally.

11. The method according to claim 10, characterized in that deoxypeganine is administered transdermally.

12-21. (Canceled)
